SEQUENCE LISTING (6 sequences)

SEQ ID NO: 1 - DNA, 720 bp, Homo sapiens
atggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtga

SEQ ID NO: 2 - DNA, 22 bp, Artificial
  Synthetic oligo
tctgtctccggtgaaggtgaag

SEQ ID NO: 3 - DNA, 21 bp, Artificial
  Synthetic olig
ggcatggcagacttgaaaaag

SEQ ID NO: 4 - DNA, 18 bp, Artificial
  Synthetic oligo
aggctgggatgcctttgt

SEQ ID NO: 5 - DNA, 22 bp, Artificial
  Synthetic oligo
gacttcacttgtggcccagata

SEQ ID NO: 6 - protein, 239 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

(19) United States
(12) Patent Application Publication
(10) Pub. No.: US 2017/0152569 A1
(43) Pub. Date: Jun. 1, 2017

(54) POLYMORPHISM IN THE BCL2 GENE DETERMINES RESPONSE TO CHEMOTHERAPY

(71) Applicant: HIDENSEQ, Shoham (IL)

(72) Inventors: Sol EFRONI, Rehovot (IL); Alona ZILBERBERG, Tel Aviv (IL); Rotem BEN-HAMO, Givatayim (IL)

(73) Assignee: HIDENSEQ, Shoham (IL)

(21) Appl. No.: 15/319,628

(22) PCT Filed: Jun. 18, 2015
(86) PCT No.: PCT/IL2015/050622

§ 371 Date: Dec. 16, 2016

Related U.S. Application Data
(60) Provisional application No. 62/014,349, filed on Jun. 19, 2014.
(60) Provisional application No. 62/120,897, filed on Feb. 26, 2015.

(51) Int. Cl. C12Q 1/68; A61K 31/337

(52) U.S. Cl. CPC C12Q 1/6886; A61K 31/337; C12Q 2600/156; C12Q 2600/106; C12Q 2600/158

(57) ABSTRACT

Provided are methods useful for determining the expected efficacy of certain chemotherapy treatments and methods of treating cancer based on said determination. In particular, provides are methods and procedures of predicting the efficacy of paclitaxel treatments in cancer patients.

### FIELD OF THE INVENTION

The present invention relates to methods useful for determining the expected efficacy of certain chemotherapy treatments and to methods of treating cancer based on said determinations.

### BACKGROUND OF THE INVENTION

Cancer, characterized by uncontrolled growth and spread of abnormal cells, is a leading cause of death in developed countries. As the average age of the population continues to rise, so do the numbers of diagnosed cases of cancer. The world health organization reports on 8.2 million deaths from cancer in the world in 2012. The most prevalent types of human cancers according to the National Cancer Institute (NCI) are bladder, breast, colon and rectal, and endometrial cancers.

Ovarian cancer is the sixth most common tumor in women. More than 200,000 new cases are diagnosed each year worldwide (6.6 new cases per 100,000). In the last two decades there have been only small improvements (20% to only 25%) in the overall survival rate of five years. Ovarian cancer, the most lethal gynecologic malignancy, has usually been treated, since the mid-1990s, with surgical debulking in combinations with platinum/taxane chemotherapy. Ovarian cancer is often diagnosed at an advanced stage because the symptoms can be vague until late-stage.

The most common types of ovarian cancer, comprising more than 95% of cases, are ovarian carcinomas. They include five main subtypes, of which high-grade serous is most common. These tumors are believed to usually initiate in the cells covering the ovaries, though some may form from the fallopian tubes. Less common types include germ cell tumors and sex cord stromal tumors. The diagnosis is confirmed by examination of a biopsy.

Another type of cancer is uterine corpus endometrial carcinoma (UCEC) that develops in cells that form the inner lining of the uterus, or the endometrium. It is one of the most common cancers of the female reproductive system among American women.

The Bcl-2 Protein Family

The Bcl-2 protein family includes mitochondrial outer membrane permeabilization (MOMP) proteins that can be either pro-apoptotic (such as, Bax, BAD, Bak and Boks) or anti-apoptotic (such as Bcl-2, Bcl-xL, and Bcl-w). The Bcl-2 family members contain at least one Bcl-2-homology (BH) domain. Though all Bcl-2 family members demonstrate membrane channel forming activity, Bcl-2 (the archetypal Bcl-2 family member) channels are cation (Ca²⁺) selective and, owing to its exclusive endoplasmic reticulum and mitochondrial membrane localization, the anti-apoptotic function of Bcl-2 is at least partly mediated by its ability to prevent calcium release from the ER and subsequent mitochondrial membrane perturbation and cytochrome c release.

The anti-apoptotic protein Bcl-2 is overexpressed in various cancers, including ovarian cancers, and contributes to drug-resistant disease, resulting in poor clinical outcome. Bcl-2 contributes to chemoresistance by stabilizing the mitochondrial membrane against apoptotic insults. Several targeted cancer therapy studies have focused on the development of agents that inhibit Bcl-2, including antisense oligonucleotides and small molecular inhibitors of Bcl-2 (Yip and Reed, Oncogene 27, 6398-6406, 2008).

Chemotherapy

Chemotherapy treatments have a range of side effects that depend on the type of medications used. The most common medications affect mainly the fast-dividing cells of the body, such as blood cells and the cells lining the mouth, stomach, and intestines. Chemotherapy related toxicities can occur acutely after administration, within hours or days, or chronically, from weeks to years.

Paclitaxel is a chemotherapeutic drug which belongs to the taxane family and is used in treating malignant diseases, such as ovarian, breast, lung and prostate cancers, as well as melanoma and other types of solid tumor cancers. Also, paclitaxel serves as an anti proliferative agent for treating conditions having undesirable cell proliferation. Though offering substantial improvement for many patients, other patients display resistance to paclitaxel.

Chemotherapy resistance is a major obstacle for the success of cancer therapy. Drug resistance can be defined by the amount of anti-cancer drug that is required to produce a given level of cell death. Clinical drug resistance can be defined either as the lack of reduction in the size of the tumor following chemotherapy or as the occurrence of clinical relapse after an initial ‘positive’ response to the treatment.

Dose fractioning, multiple cycles and additional chemotherapeutic agents have shown limited effect on outcome in the case of ovarian cancer treatment. Moreover, chemotherapy treatments cause tremendous suffering and pain to patients.

Thus, it would be highly advantageous to have methods of predicting the efficiency of chemotherapy drugs and to individualize the treatments so as to improve effectiveness, and thereby reduce toxicity as well as costs of treatments.

### SUMMARY OF THE INVENTION

The present invention provides methods for determining the predicted efficacy of certain chemotherapeutic drugs in cancer patients. According to some aspects the present invention identifies a silent polymorphism in the human BCL2 gene that, while it does not affect the protein sequence, does appear to be correlated with over expression of the encoded protein. According to some aspects of the present invention it is now disclosed that subjects bearing this genetic variant may be less susceptible to certain types of chemotherapy. According to some aspects, subjects bearing this genetic marker may require treatment protocols that utilize additional drugs or alternative drug regimes including those that do not target Bcl-2.

According to some particular embodiments the present invention provides methods for evaluating the efficacy of taxol in the treatment of cancer patients. According to particular embodiments the principles of the invention are exemplified in ovarian cancer patients. According to additional embodiments the principles of the invention are exemplified in uterine corpus endometrial carcinoma, head and neck squamous cell carcinoma, bladder urothelial carcinoma, cervical squamous cell carcinoma, esophageal carcinoma, lung adenocarcinoma, lung squamous carcinoma, skin cutaneous melanoma, stomach adenocarcinoma, and cutaneous melanoma.

An accurate forecasting of the response of a cancer patient to chemotherapy such as paclitaxel enables the planning of suitable and individualized chemotherapy regimens.

The present invention is based in part on the unexpected discovery that patients having a specific genotypic variant of the BCL2 gene exhibit non-responsiveness for paclitaxel treatment. The inventors of the present invention have found a direct correlation between having a G allele at SNP rs1801018 and non-responsiveness to paclitaxel treatments. Patients having a G allele at SNP rs1801018 were found to respond to alternative drugs such as docetaxel.

Without wishing to be bound by any particular theory or mechanism of action, this difference in chemotherapeutic efficacy responsiveness may be attributed to the specific genotypic variant that produces a more stable BCL2 transcript, which results in higher amounts of Bcl-2 protein.

According to a first aspect, the present invention provides a method of selecting an agent suitable for treating cancer in a subject, comprising: providing a sample of genetic material from the subject; and determining the single nucleotide polymorphism (SNP) rs1801018 allele in the sample of the subject; wherein homozygosity for the A allele at SNP rs1801018 is indicative of paclitaxel as a suitable anti-cancer agent, and the presence of at least one G allele at SNP rs1801018 is indicative of potential resistance to paclitaxel, necessitating an alternative agent as the suitable anti-cancer agent.

According to some embodiments, the cancer is a solid cancer. According to certain embodiments, the cancer is selected from the group consisting of ovarian cancer, uterine corpus endometrial carcinoma (UCEC), head and neck squamous cell carcinoma (HNSC), lung cancer, colon cancer, breast cancer, pancreatic cancer, prostate cancer, chronic myelogenous leukemia, and melanoma. According to additional embodiments, the cancer is selected from the group consisting of ovarian cancer, uterine corpus endometrial carcinoma (UCEC), and head and neck squamous cell carcinoma (HNSC), bladder urothelial carcinoma (BLCA), cervical squamous cell carcinoma (CESC), esophageal carcinoma (ESCA), lung adenocarcinoma (LUAD), lung squamous carcinoma (LUSC), skin cutaneous melanoma (SKCM), stomach adenocarcinoma (STAD), and cutaneous melanoma (UCS). According to certain embodiments, the cancer is selected from the group consisting of ovarian cancer, uterine corpus endometrial carcinoma (UCEC), and head and neck squamous cell carcinoma (HNSC). According to additional embodiments, the cancer is ovarian cancer. Each possibility represents a separate embodiment of the present invention.

According to some embodiments, the cancer is a type of cancer considered amenable for treatment with paclitaxel.

According to some embodiments, the selection of an alternative agent is based on further determination of an over-expression level of at least one gene of the set specified hereinbelow in the subject compared to a non-cancer control subject. According to additional embodiments, the selection of an alternative agent is based on further determination of over-expression levels of at least 2, 5, 10, 15, 20, 25, or 29 genes in the subject compared to a non-cancer subject. Each possibility represents a separate embodiment of the invention.

According to certain embodiments, the selection of an alternative agent is based on further determination of a linear combination of the expression levels of at least two genes specified hereinbelow in the subject. According to additional embodiments, the selection of an alternative agent is based on further determination using of a linear combination of the expression levels of the genes specified hereinbelow in the subject.

According to some embodiments, the determination using of a linear combination of the expression levels of the genes is compared to non-cancer control subject. In other embodiments, the control is a predetermined value.

According to certain embodiments, at least one gene is selected from the group consisting of TNFRSF17, MZB1/MGC29506, PLA2G2D, PA2G4, MCAT, PAFAH2, SLAMF7, NSF, PPP2R1A, CKMT2, IGHG1, CCDC56, HGD, ASB8, IGL@, CDH16, EFHD1, CES2, CDK10, LAX1, IGLV460, ATXN10, Clorf216, DNAJB12, COL4A6, HABP4, IGLV657, LOC652494, and DDX19A. Each possibility represents a separate embodiment of the present invention.

According to some embodiments, the alternative anti-cancer therapy is a non-taxane. According to various embodiments the non-taxane alternative anti-cancer agent is selected from the group consisting of an antimetabolite, a mitotic inhibitor, a topoisomerase inhibitor, a topoisomerase II inhibitor, an asparaginase, an alkylating agent, an antitumor antibiotic, and combinations thereof. Each possibility represents a separate embodiment of the invention. According to another embodiment the alternative or additional anticancer agent is an alternative taxane. According to this embodiment the alternative taxane is docetaxel.

According to some embodiments, the antimetabolite is selected from the group consisting of cytarabine, gludarabine, fluorouracil, mercaptopurine, methotrexate, thioguanine, gemcitabine, and hydroxyurea. According to some embodiments, the mitotic inhibitor is selected from the group consisting of vincristine, vinblastine, and vinorelbine. According to some embodiments, the topoisomerase inhibitor is selected from the group consisting of topotecan and irenotecan. According to some embodiments, the alkylating agent is selected from the group consisting of busulfan, carmustine, lomustine, chlorambucil, cyclophosphamide, cisplatin, carboplatin, ifosamide, mechlorethamine, melphalan, thiotepa, dacarbazine, and procarbazine. According to some embodiments, the antitumor antibiotic is selected from the group consisting of bleomycin, dactinomycin, daunorubicin, doxorubicin, idarubicin, mitomycin, mitoxantrone, and plicamycin. According to some embodiments, the topoisomerase II is selected from the group consisting of etoposide and teniposide. Each possibility represents a separate embodiment of the present invention.

According to some particular embodiments, the alternative anti-cancer agent is selected from the group consisting of bevacizumab, carboplatin, cyclophosphamide, doxorubicin hydrochloride, gemcitabine hydrochloride, topotecan hydrochloride, thiotepa, and combinations thereof. Each possibility represents a separate embodiment of the present invention.

According to some embodiments, the sample from the subject comprises a nucleic acid polymer. According to certain embodiments, the sample comprises DNA. According to additional embodiments, the sample comprises RNA.

According to some embodiments, determining the allele at single nucleotide polymorphism (SNP) rs1801018 is performed by a technique selected from the group consisting of: analysis using a whole genome SNP chip, single-stranded conformational polymorphism (SSCP) assay, restriction fragment length polymorphism (RFLP), automated fluorescent sequencing; clamped denaturing gel electrophoresis (CDGE); denaturing gradient gel electrophoresis (DGGE), restriction enzyme analysis, chemical mismatch cleavage (CMC), RNase protection assays, use of polypeptides that recognize nucleotide mismatches, allele-specific PCR, sequence analysis, and SNP genotyping. Each possibility represents a separate embodiment of the invention. According to certain embodiments, the determining the allele at single nucleotide polymorphism comprises DNA amplification.

According to additional embodiments, the method of the invention comprises the step of carrying out at least one gene amplification using a pair of primers selected from the group consisting of primers capable of amplifying a region of the BCL2 gene comprising SNP rs1801018. According to further additional embodiments, the method of the invention further comprises analyzing the amplified product by comparison with the wild type sequence of BCL2 gene.

According to some embodiments, the sample is blood, serum, skin tissue, or saliva.

According to some embodiments, the sample is selected from the group consisting of: in vitro sample, ex vivo sample, and in situ sample.

According to some embodiments, the subject is a human subject.

According to some embodiments, the subject is already being treated with a chemotherapy drug.

According to an additional aspect, the present invention provides a method of treating cancer in a subject in need of such treatment, comprising:
(i) determining the genotype of a single nucleotide polymorphism (SNP) rs1801018 in a sample of the subject; and
(ii) administering paclitaxel to the subject when the sample is homozygous for the A allele of SNP rs1801018, or administering an alternative anti-cancer agent to the subject when at least one G allele of the SNP rs1801018 is present.

According to some embodiments, the cancer is a solid cancer. According to certain embodiments, the cancer is selected from the group consisting of ovarian cancer, uterine corpus endometrial carcinoma (UCEC), head and neck squamous cell carcinoma (HNSC), lung cancer, colon cancer, breast cancer, pancreatic cancer, prostate cancer, chronic myelogenous leukemia, and melanoma. According to additional embodiments, the cancer is selected from the group consisting of ovarian cancer, uterine corpus endometrial carcinoma (UCEC), and head and neck squamous cell carcinoma (HNSC), bladder urothelial carcinoma (BLCA), cervical squamous cell carcinoma (CESC), esophageal carcinoma (ESCA), lung adenocarcinoma (LUAD), lung squamous carcinoma (LUSC), skin cutaneous melanoma (SKCM), stomach adenocarcinoma (STAD), and cutaneous melanoma (UCS). According to certain embodiments, the cancer is selected from the group consisting of ovarian cancer, uterine corpus endometrial carcinoma (UCEC), and head and neck squamous cell carcinoma (HNSC). According to additional embodiments, the cancer is ovarian cancer. Each possibility represents a separate embodiment of the invention.

According to some embodiments, the cancer is a type of cancer considered amenable for treatment with paclitaxel.

The alternative anti-cancer therapy is as described hereinabove.

According to some embodiments, the sample comprises a nucleic acid polymer. According to certain embodiments, the sample comprises DNA. According to additional embodiments, the sample comprises RNA.

According to some embodiments, determining the allele at single nucleotide polymorphism (SNP) rs1801018 is performed by a technique selected from the group consisting of: analysis using a whole genome SNP chip, single-stranded conformational polymorphism (SSCP) assay, restriction fragment length polymorphism (RFLP), automated fluorescent sequencing; clamped denaturing gel electrophoresis (CDGE); denaturing gradient gel electrophoresis (DGGE), restriction enzyme analysis, chemical mismatch cleavage (CMC), RNase protection assays, use of polypeptides that recognize nucleotide mismatches, allele-specific PCR, sequence analysis, and SNP genotyping. Each possibility represents a separate embodiment of the invention.

According to some embodiments, the subject is a human subject.

According to some embodiments, the subject is already being treated with a chemotherapy drug.

According to some embodiments, the method comprises administering or performing at least one additional anti cancer therapy. According to certain embodiments, the additional anticancer therapy is surgery, chemotherapy, radiotherapy, or immunotherapy.

According to some embodiments, the anti-cancer agent is administered by a route selected from the group consisting of oral, intravenous, intramuscular and subcutaneous injection.

According to some embodiments, the method of treating further comprises a step of determining the expression of at least one gene of the set specified hereinbelow, wherein an alternative agent is administered when the gene is over-expressed compared to its expression in non-cancer subjects. According to some embodiments, the method of treating further comprises a step of determining the expression of at least 1, 5, 10, 15, 20, 25, or 29 genes of the set specified hereinbelow, wherein an alternative agent other than a paclitaxel is administered when said gene or genes are over-expressed when compared to expression in a non-cancer control subject. Each possibility represents a separate embodiment of the invention.

According to certain embodiments, the method of treating further comprises the step of determination a linear combination of expression levels of at least two genes of the set specified hereinbelow, wherein an alternative agent is administered when the at least two genes are over-expressed in the subject compared to the expression in non-cancer subjects.

According to additional embodiments, the method of treating further comprises the step of determination a linear combination of expression levels of the genes specified hereinbelow, wherein an alternative agent is administered when the genes are over-expressed in the subject compared to the expression in non-cancer subjects.

According to some embodiments, the determination using of a linear combination of the expression levels of the genes is compared to a non-cancer control subject. In other embodiments, the control is a predetermined value.

According to certain embodiments, at least one gene is selected from the group consisting of TNFRSF17, MZB1/MGC29506, PLA2G2D, PA2G4, MCAT, PAFAH2, SLAMF7, NSF, PPP2R1A, CKMT2, IGHG1, CCDC56, HGD, ASB8, IGL@, CDH16, EFHD1, CES2, CDK10, LAX1, IGLV460, ATXN10, Clorf216, DNAJB12, COL4A6, HABP4, IGLV657, LOC652494, and DDX19A. Each possibility represents a separate embodiment of the present invention.

According to additional embodiments, the agent is administered in a stent.

According to additional aspect, the present invention provides a method of selecting an agent suitable for treating a condition amenable for paclitaxel treatment in a subject comprising: providing a sample of genetic material from the subject; determining a single nucleotide polymorphism (SNP) rs1801018 allele in the sample of the subject, wherein homozygosity for the A allele at SNP rs1801018 is indicative of paclitaxel as a suitable agent, and the presence of at least one G allele at SNP rs1801018 is indicative of the need for an alternative agent as the suitable agent.

According to some embodiments, the agent is used for preventing and/or reducing and/or treating undesired cell proliferation. According to additional embodiments, the agent is used for preventing and/or reducing and/or treating neointimal hyperplasia. Each possibility represents a separate embodiment of the invention. According to specific embodiments, the agent is administered to patients with coronary artery lesions. According to other embodiments the condition is cancer.

According to some embodiments, the agent is administered in a stent.

According to additional aspect, the present invention provides an anti-cancer agent for use in treating cancer, wherein said use comprises determining the single nucleotide polymorphism (SNP) rs1801018 allele in a sample of a subject afflicted with cancer, wherein homozygosity for the A allele at SNP rs1801018 is indicative of using paclitaxel as the anti-cancer agent, and the presence of at least one G allele at SNP rs1801018 is indicative of using an alternative agent as the anti-cancer agent.

According to additional aspect, the present invention provides a method of treating a condition amenable for paclitaxel treatment in a subject in need of such treatment, comprising:
(i) determining the genotype of a single nucleotide polymorphism (SNP) rs1801018 in a sample of the subject; and
(ii) administering paclitaxel to the subject when the sample is homozygous for the A allele of the SNP rs1801018, or administering alternative agent to the subject when at least one G allele of the SNP rs1801018 is present.

According to some embodiments, the agent or paclitaxel is administered in a stent.

According to some embodiments, the agent is used for preventing and/or reducing and/or treating undesired cell proliferation. According to additional embodiments, the agent is used for preventing and/or reducing and/or treating neointimal hyperplasia. Each possibility represents a separate embodiment of the invention. According to specific embodiments, the agent is administered to patients with coronary artery lesions. According to other embodiments the condition is cancer.

The alternative agent is as described hereinabove.

Other objects, features and advantages of the present invention will become clear from the following description and drawings.

### BRIEF DESCRIPTION OF THE FIGURES

FIGS. 1A-1J illustrate the correlation between SNP rs1801018 allele type and paclitaxel treatment efficacy.

FIG. 1A depicts the frequency of various SNPs in BCL2 and TUBB1 (tubulin) genes in cancer patients.

FIG. 1B shows there is no correlation between having SNP rs6070697 and the anti-cancer treatment regimen.

FIG. 1C shows a scheme of chromosome 18 and SNP rs1801018 position.

FIG. 1D illustrates the correlation between chemotherapy regimen to ovarian cancer (OV) patients and having a certain allele of SNP rs1801018.

FIG. 1E summarizes the correlation between chemotherapy regimen to ovarian cancer patients and having a certain allele of SNP rs1801018.

FIG. 1F illustrates the correlation between chemotherapy regimen to uterine corpus endometrial carcinoma (UCEC) patients and having certain allele of SNP rs1801018.

FIG. 1G summarizes the correlation between chemotherapy regimen to uterine corpus endometrial carcinoma (UCEC) patients and having certain allele of SNP rs1801018.

FIG. 1H illustrates the correlation between chemotherapy regimen to head and neck squamous cell carcinoma (HNSC) patients and having certain allele of SNP rs1801018.

FIG. 1I summarizes the correlation between chemotherapy regimen to head and neck squamous cell carcinoma (HNSC) patients and having certain allele of SNP rs1801018.

FIG. 1J describes a validation test for the correlation between the SNP variant and the treatment regimen. The test was performed for 8 different cancers as indicated.

FIGS. 2A-2D illustrate the effect of SNP rs1801018 on the BCL2 mRNA stability and expression, and Bcl-2 protein expression.

FIG. 2A shows relative BCL2 mRNA expression levels in HeyA8 cells transfected with green fluorescent protein (GFP) empty vector, or one of three types of GFP-BCL2 variants (wt, rs1801018, and random).

FIG. 2B shows the relative stability of mRNA transcripts. HeyA8 cells were transfected with GFP empty vector (GFP), or one of three types of GFP-BCL2 variants (wt (BCL2-WT), rs1801018 (BCL2-Mut), and random (BCL2-Rand)). Actinomycin D was added 24 hours later, and BCL2 expression levels were examined at the indicated times.

FIG. 2C shows a relative expression of endogenous Bcl-2 (BCL2) and GFP-Bcl-2 (GFP-BCL2) in HeyA8 cells transfected with GFP empty vector, or one of three types of GFP-BCL2 variants (wt, rs1801018, and random). The figure shows representative western blot assays with anti-Bcl-2 or anti-actin antibodies.

FIG. 2D summarizes the relative protein expression levels in HeyA8 cells transfected with one of three GFP-BCL2 variants (wt, rs1801018, and random).

FIG. 3 shows the effect of paclitaxel on cells expressing different GFP-BCL2 variants (wild type (BCL2-WT), rs1801018 (BCL2-Mut), or random SNP (BCL2-Rand)).

FIG. 4 shows the effect of transfecting cells with different BCL2 mRNA concentrations on cells sensitivity to paclitaxel treatment.

### DETAILED DESCRIPTION OF THE INVENTION

The present invention allows for the determination of whether a chemotherapeutic agent will be effective in treating cancer when administered to a patient. In particular, the present invention provides methods of determining the predicted efficacy of certain chemotherapeutic treatments in cancer patients. In other aspects, the present invention provides methods of treating cancer based on selecting a suitable chemotherapy agent according to an individual SNP genotype.

Paclitaxel is considered a first line treatment for a wide variety of cancers including advanced ovarian carcinoma. Therefore, methods that determine in advance the efficacy of paclitaxel treatments are of great importance.

The invention is based on the unexpected discovery that there is a direct correlation between non-responsiveness to paclitaxel and a specific genotypic variant of the BCL2 gene (resulting in A to G substitution at position 21 of the nucleic acid sequence set forth in SEQ ID NO: 1). In Korean populations the genotypic variant, refers to as synonymous SNP rs1801018 was found to be correlated with susceptibility to papillary thyroid cancer. The G allele of BCL2 is associated with the multifocality and bilaterality of papillary thyroid cancer (PTC) (Eun et al. Clin Exp Otorhinolaryngol. 2011 September; 4(3):149-54).

It is now disclosed for the first time that the SNP rs1801018 is highly correlated with the responsiveness of variety of cancer patients to paclitaxel treatments.

Dose fractioning, multiple cycles and an additional chemotherapeutic agent have shown limited effect in the case of ovarian cancer treatment. Acquiring evaluation tools to individualize patients' treatment would reduce toxicity as well as costs of treatment. The inventors of the present invention have found that ovarian cancer patients bearing the G allele of rs1801018 possess high resistance to paclitaxel treatments. The present invention establishes an improved personalized therapy regimen for ovarian cancer patients. For example, a patient having a G allele may be excluded from paclitaxel single line treatments and be treated with another or combinations of other anti-cancer drugs. The present invention further provides that the SNP rs1801018 correlates with paclitaxel efficacy in additional cancer types, thus establishing a general tool for predicting the responsiveness of cancer patients to paclitaxel.

Without wishing to be bound by any particular theory or mechanism of action the difference between the variants may be attributed to a structurally change of the transcript encoded by the BCL2 gene. The alternation of the mRNA structure leads to higher stability of the BCL2 transcript, which results in higher amounts of the Bcl-2 protein.

As used herein, Bcl-2 means the human Bcl-2 (B-cell lymphoma 2) which is an apoptosis regulator protein that has two isoforms. The present invention refers to the Bcl-2 encoded by transcript variant alpha (GenBank Accession No. NM_000633.2). According to some embodiments, the Bcl-2 protein sequence is as set forth in SEQ ID NO:6.

Bcl-2 acts as a gatekeeper of the permeability transition pore complex (PTPC), a mitochondrial polyprotein complex, which participates in the regulation of matrix Ca²⁺, pH, mitochondrial membrane potential (ΔΨm), and volume and functions as a Ca²⁺-, voltage-, pH-, and redox-gated channel with several levels of conductance and little, if any, ion selectivity. Moreover, it is regulated by the antiapoptotic oncoproteins Bcl-2 and Bcl-XL, which stabilize mitochondrial membranes, and by the proapoptotic Bcl-2 analogue Bax, which disrupts the ΔΨm.

In the presence of paclitaxel, Bcl-2 function of blocking the PTPC is abolished and reversed, with it instead easing the opening of the channel and the consequent reduction in ΔΨm. Without wishing to be bound by any particular theory or mechanism, elevated amount of the Bcl-2 protein in G allele genotypic variants confers the resistance to paclitaxel phenomenon in the patients.

Paclitaxel or ((2α,4α,5β,7β,10β,13α)-4,10-Bis(acetyloxy)-13-{[(2R,3S)-3-(benzoylamino)-2-hydroxy-3-phenylpropanoyl]oxy}-1,7-dihydroxy-9-oxo-5,20-epoxytax-11-en-2-yl benzoate) is a mitotic inhibitor used in cancer chemotherapy. Paclitaxel is used to treat cancers such as, without limiting, lung, ovarian, breast, and head and neck cancers. In addition, paclitaxel is used for treating conditions characterized by undesirable cell proliferation.

Docetaxel or 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate} is a member of the taxane drug class. It interferes with microtubules, serving as an anti-mitotic chemotherapy medicament.

According to one aspect, the present invention provides a method of selecting an agent suitable for treating a cancer, the method comprises:

(i) obtaining a nucleic acid-containing sample from a subject afflicted with cancer;

(ii) determining the single nucleotide polymorphism (SNP) rs1801018 allele in said sample;

wherein homozygosity for the A allele at SNP rs1801018 is indicative of paclitaxel as the suitable agent, and the presence of at least one G allele at SNP rs1801018 is indicative of an alternative agent as the suitable anti-cancer agent.

According to another aspect, the present invention provides a method of selecting an agent suitable for treating cancer in a subject, comprising determining the single nucleotide polymorphism (SNP) rs1801018 allele in a sample of the subject; wherein homozygosity for the A allele at SNP rs1801018 is indicative of paclitaxel as a suitable anti-cancer agent, and the presence of at least one G allele at SNP rs1801018 is indicative of an alternative agent as the suitable anti-cancer agent.

According to additional aspect, the present invention provides a method of treating cancer in a subject in need of such treatment, comprising:
(i) determining the genotype of a single nucleotide polymorphism (SNP) rs1801018 in a sample of the subject; and
(ii) administering paclitaxel to the subject when the sample is homozygous for the A allele of SNP rs1801018, or administering alternative anti-cancer agent to the subject when at least one G allele of the SNP rs1801018 is present.

According to another aspect, the present invention provides a method of treating cancer in a subject in need of such treatment, comprising:
(i) obtaining a nucleic acid containing sample from said subject;
(ii) determining the genotype of a single nucleotide polymorphism (SNP) rs1801018 in said sample; and
(iii) administering paclitaxel to the subject wherein the sample is homozygous for the A allele of SNP rs1801018, or administering alternative anti-cancer agent to the subject when at least one G allele of the SNP rs1801018 is present.

According to additional aspect, the present invention provides an anti-cancer agent for use in treating cancer, wherein said use comprises determining the single nucleotide polymorphism (SNP) rs1801018 allele in a sample of a subject afflicted with cancer, wherein homozygosity for the A allele at SNP rs1801018 is indicative of using paclitaxel as the anti-cancer agent, and the presence of at least one G allele at SNP rs1801018 is indicative of using an alternative agent as the anti-cancer agent.

According to additional aspect, the present invention provides an anti-cancer agent for use in treating cancer, wherein said use is preceded by the step of determining the single nucleotide polymorphism (SNP) rs1801018 allele in a sample of a subject afflicted with cancer, wherein homozygosity for the A allele at SNP rs1801018 is indicative of paclitaxel as the anti-cancer agent for use in treating said cancer, and the presence of at least one G allele at SNP rs1801018 is indicative of an alternative agent as the anti-cancer agent for use in treating said cancer.

According to additional aspect, the present invention provides a method of selecting an agent suitable for treating cancer in a subject, comprising: obtaining a sample of genetic material from the subject; determining the single nucleotide polymorphism (SNP) rs1801018 allele in the sample of the subject; wherein homozygosity for the A allele at SNP rs1801018 is indicative of paclitaxel as a suitable anti-cancer agent, and homozygosity for the G allele at SNP rs1801018 is indicative of an alternative agent as the suitable anti-cancer agent.

According to additional aspect, the present invention provides a method of selecting an agent suitable for treating cancer in a subject, comprising: obtaining a sample of genetic material from the subject; determining the single nucleotide polymorphism (SNP) rs1801018 allele in the sample of the subject; wherein homozygosity for the A allele at SNP rs1801018 is indicative of paclitaxel as a suitable anti-cancer agent, and homozygosity for the G allele at SNP rs1801018 is indicative of a docetaxel as the suitable anti-cancer agent.

According to another aspect, the present invention provides a method of treating cancer in a subject in need of such treatment, comprising:
(i) obtaining a nucleic acid containing sample from said subject;
(ii) determining the genotype of a single nucleotide polymorphism (SNP) rs1801018 in said sample; and
(iii) administering paclitaxel to the subject wherein the sample is homozygous for the A allele of SNP rs1801018, or administering docetaxel to the subject when at least one G allele of the SNP rs1801018 is present.

According to some embodiments, the cancer is a type of cancer considered amenable for treatment with paclitaxel.

The methods of the invention are suitable for any condition that is considered to be amenable for treatment with paclitaxel. Paclitaxel is used inter alia as part of the treatment for coronary artery lesions. Stent containing paclitaxel is used to prevent cell proliferation and neointimal hyperplasia. Neointimal hyperplasia is proliferation and migration of vascular smooth muscle cells primarily in the tunica intima, resulting in the thickening of arterial walls and decreased arterial lumen space. Neointimal hyperplasia is the major cause of restenosis after percutaneous coronary interventions such as stenting or angioplasty.

According to additional aspect, the present invention provides a method of selecting an agent suitable for treating a condition amenable for paclitaxel treatment in a subject comprising: providing a sample of genetic material from the subject; determining a single nucleotide polymorphism (SNP) rs1801018 allele in the sample of the subject, wherein homozygosity for the A allele at SNP rs1801018 is indicative of paclitaxel as a suitable agent, and the presence of at least one G allele at SNP rs1801018 is indicative of the need for an alternative agent as the suitable agent.

According to some embodiments, the agent is used for preventing and/or reducing and/or treating undesired cell proliferation. According to additional embodiments, the agent is used for preventing and/or reducing and/or treating neointimal hyperplasia. Each possibility represents a separate embodiment of the invention. According to specific embodiments, the agent is administered to patients with coronary artery lesions.

According to some embodiments, the agent is administered in a stent.

According to additional aspect, the present invention provides a method of treating a condition amenable for paclitaxel treatment in a subject in need of such treatment, comprising:
(i) determining the genotype of a single nucleotide polymorphism (SNP) rs1801018 in a sample of the subject; and
(ii) administering paclitaxel to the subject when the sample is homozygous for the A allele of the SNP rs1801018, or administering alternative agent to the subject when at least one G allele of the SNP rs1801018 is present.

According to some embodiments, the agent or paclitaxel is administered in a stent.

According to some embodiments, the agent is used for preventing and/or reducing and/or treating undesired cell proliferation. According to additional embodiments, the agent is used for preventing and/or reducing and/or treating neointimal hyperplasia. Each possibility represents a separate embodiment of the invention. According to specific embodiments, the agent is administered to patients with coronary artery lesions.

The alternative agent is as described hereinabove.

### DEFINITIONS

The terms “non-response” or “non-responsiveness” in the context of paclitaxel and within the meaning of the present invention refer to a lack of clinical response, a negative clinical response, or not sufficient clinical response to the treatment. In some embodiments, the subject is resistant to paclitaxel.

The term “amenable for paclitaxel treatment” refers to a condition that is known to be treated by paclitaxel such as ovarian cancer. The term further refers to cancer, wherein treatment with paclitaxel may have a potentially beneficial effect. The term further refers to any condition characterized by cell proliferation and known to be treated with paclitaxel.

As referred to herein, the term “treating” is directed to administering a composition, which comprises at least one active agent, effective to ameliorate symptoms associated with a disease, to lessen the severity or cure the disease. In some embodiments, treating results in a reduction in tumor size. In additional embodiments, treating results in the decrease of tumor growth and/or decrease or inhibit of tumor metastasis.

The term “allele” as used herein refers to different forms of a gene, composed of one or more single nucleotide polymorphism. “A allele” or “G allele” as used herein refer to different forms of the SNP rs1801018 as reflected at position 21 in the mRNA product sequence set forth in SEQ ID NO:1.

The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Typically, a reference sequence is referred to for a particular sequence. A reference sequence as used herein is the wild type allele. A “variant” sequence, as used herein, refers to a wild type sequence or a sequence that differs from the wild type sequence but is otherwise substantially similar. For example, different alleles at the single nucleotide polymorphic sites described herein are variants. Variants can include changes that affect a polypeptide folding structure, expression levels and/or have no effect on polypeptide. Patients or cells can be homozygous, having the two wild type alleles or one of the other variant alleles. Patients or cells can be also heterozygous, having a wild type allele and one of the other variants allele.

### Determining Single Nucleotide Polymorphism

Detecting specific polymorphic markers can be accomplished by methods known in the art for detecting sequences at polymorphic sites. For example, standard techniques for genotyping for the presence of SNPs can be used, such as fluorescence-based techniques (e.g., Chen, X. et al., Genome Res. 9(5): 492-98 (1999); Kutyavin et al., Nucleic Acid Res. 34:e128 (2006)), utilizing PCR, LCR, Nested PCR and other techniques for nucleic acid amplification. Specific commercial methodologies available for SNP genotyping include, but are not limited to, TaqMan genotyping assays and SNPlex platforms (Applied Biosystems), gel electrophoresis, mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumina GoldenGate and Infinium assays), array tag technology (e.g., Parallele), and endonuclease-based fluorescence hybridization technology (Invader; Third Wave). Some of the available array platforms include Affymetrix SNP Array 6.0 and Illumina CNV370-Duo and 1M BeadChips. Thus, by use of these or other methods available to the person skilled in the art, one or more alleles at polymorphic markers, can be identified.

In some embodiments, determining the allele at single nucleotide polymorphism (SNP) rs1801018 is performed by a technique selected from the group consisting of: analysis using a whole genome SNP chip, single-stranded conformational polymorphism (SSCP) assay, restriction fragment length polymorphism (RFLP), automated fluorescent sequencing; clamped denaturing gel electrophoresis (CDGE); denaturing gradient gel electrophoresis (DGGE), restriction enzyme analysis, chemical mismatch cleavage (CMC), RNase protection assays, use of polypeptides that recognize nucleotide mismatches, allele-specific PCR, sequence analysis, and SNP genotyping. Each possibility represents a separate embodiment of the invention. In one embodiment, the single nucleotide polymorphism is identified by the direct sequencing of a given gene by the use of gene-specific oligonucleotides as sequencing primers.

The sample can be any nucleic acid containing sample. In some embodiments, the sample is selected from the group consisting of: peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a tumor tissue or a suspected tumor tissue, a thin layer cytological sample, a fine needle aspirate sample, a bone marrow sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, and a paraffin embedded tissue sample.

### Malignant Diseases

The invention further provides a method for treating cancer.

The terms “cancer”, “malignant disease”, “tumor”, and the like are used interchangeably herein to refer to conditions characterized by cells which exhibit relatively autonomous growth, so that they exhibit an aberrant growth phenotype characterized by a significant loss of control of cell proliferation.

In some embodiments, the cancer is selected from the group consisting of: adrenocortical carcinoma, anal cancer, bladder cancer, brain tumor, brain stem glioma, brain tumor, cerebellar astrocytoma, cerebral astrocytoma, ependymoma, medulloblastoma, supratentorial primitive neuroectodermal, pineal tumors, hypothalamic glioma, breast cancer, carcinoid tumor, carcinoma, cervical cancer, colon cancer, endometrial cancer, esophageal cancer, extrahepatic bile duct cancer, ewings family of tumors (PNET), extracranial germ cell tumor, eye cancer, intraocular melanoma, gallbladder cancer, gastric cancer, germ cell tumor, extragonadal germ cell tumor, gestational trophoblastic tumor, head and neck cancer, hypopharyngeal cancer, islet cell carcinoma, laryngeal cancer, leukemia, acute lymphoblastic leukemia, oral cavity cancer, liver cancer, lung cancer, small cell lymphoma, central nervous system (primary) lymphoma, cutaneous T-cell lymphoma, hodgkin's disease, non-hodgkin's disease, malignant mesothelioma, melanoma, merkel cell carcinoma, metasatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian epithelial cancer, ovarian germ cell tumor, ovarian low malignant potential tumor, pancreatic cancer, exocrine pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cell cancer, salivary gland cancer, sezary syndrome, skin cancer, cutaneous T-cell lymphoma, skin cancer, kaposi's sarcoma, melanoma, small intestine cancer, soft tissue sarcoma, testicular cancer, thymoma, malignant, thyroid cancer, urethral cancer, uterine cancer, sarcoma, vaginal cancer, vulvar cancer, and wilms' tumor. Each possibility represents a separate embodiment of the invention.

According to additional embodiments, the cancer is selected from the group consisting of ovarian cancer, uterine corpus endometrial carcinoma (UCEC), and head and neck squamous cell carcinoma (HNSC), bladder urothelial carcinoma (BLCA), cervical squamous cell carcinoma (CESC), esophageal carcinoma (ESCA), lung adenocarcinoma (LUAD), lung squamous carcinoma (LUSC), skin cutaneous melanoma (SKCM), stomach adenocarcinoma (STAD), and cutaneous melanoma (UCS). Each possibility represents a separate embodiment of the invention.

The anti-cancer agents of the invention can be administered by any suitable administration route as know in the art. They should be administered under the supervision of a physician experienced in the use of cancer chemotherapeutic agents.

According to some embodiments, the anti-cancer agent is administered in a route selected from the group consisting of intravenous, intraperitoneal, subcutaneous, intramuscular, transdermal or oral. According to exemplary embodiments, the anti-cancer agent is administered intravenously. Each possibility represents a separate embodiment of the invention.

In another aspect, the present invention provides a kit for selecting a suitable anti-cancer agent for a subject, comprising:
(i) means for determining the genotype at SNP rs1801018 of said subject; and
(ii) written instruction for selecting a suitable anti-cancer agent, wherein homozygosity for the A alleles of rs1801018 indicates that paclitaxel is a suitable agent for treating said subject, and having at least one G allele of rs1801018 indicates that an alternative agent should be used.

A genome-wide study of human gene expression in ovarian cancer has revealed that 29 genes are highly associated with non-responsiveness to paclitaxel. A linear combination of expression levels of these genes was found to be correlated with a lack of response to paclitaxel in cancer patients.

As used herein, the term “linear correlation” refers to the comparison of the calculated sum of expression level of genes of at least two subjects each represented by a parameter calculated as the sum of multiplying each gene expression value with a different constant.

According to some embodiments, the selection of an alternative agent is based on further determination of a linear combination of the expression levels of at least one gene of the set specified herein below in the subject compared to a non-cancer control subject.

According to certain embodiment, the at least one gene is selected from the group set forth in Table 1.

TABLE 1
A list of human genes. The linear combination of expression
levels of the genes in the list predicts responsiveness
to paclitaxel in cancer patients.

| | Gene Symbol | Description |
|---|---|---|
| 1 | TNFRSF17 | Tumor Necrosis Factor Receptor, B Cell |
| | | Maturation Antigen |
| 2 | MZB1/MGC29506 | Marginal Zone B And B1 Cell Specific Protein |
| 3 | PLA2G2D | Phospholipase A2 |
| 4 | PA2G4 | Cell Cycle Protein P382G4 Homolog |
| 5 | MCAT | Mitochondrial Malonyl CoA: ACP |
| 6 | PAFAH2 | Platelet Activating Factor Acetylhydrolase |
| 7 | SLAMF7 | mediates NK cell activation |
| 8 | NSF | Vesicular Fusion Protein |
| 9 | PPP2R1A | implicated in the negative control of cell |
| | | growth and division |
| 10 | CKMT2 | responsible for the transfer of high energy |
| | | phosphate from mitochondria to the cytosolic |
| | | carrier, creatine |
| 11 | IGHG1 | Immunoglobulin Heavy Constant Gamma 1 |
| 12 | CCDC56 | Mitochondrial Translation Regulation |
| | | Assembly Intermediate Of Cytochrome C |
| 13 | HGD | catabolism of the amino acids tyrosine and |
| | | phenylalanine |
| 14 | ASB8 | Ankyrin Repeat And SOCS Box Containing 8 |
| 15 | IGL@ | Immunoglobulin lambda over expressed in B |
| | | cells and lymph nodes |
| 16 | CDH16 | Kidney specific Cadherin |
| 17 | EFHD1 | displays increased expression during neuronal |
| | | differentiation |
| 18 | CES2 | participates in fatty acyl and cholesterol ester |
| | | metabolism, and may play a role in the blood |
| | | brain barrier system. |
| 19 | CDK10 | Cell Division Protein Kinase |
| 20 | LAX1 | Linker For Activation Of X Cells |
| 21 | IGLV460 | Immunoglobulin lambda |
| 22 | ATXN10 | neuron survival, neuron differentiation, and |
| | | neuritogenesis |
| 23 | C1orf216 | — |
| 24 | DNAJB12 | HSP40 |
| 25 | COL4A6 | Collagen |
| 26 | HABP4 | remodeling of chromatin and the regulation of |
| | | transcription |
| 27 | IGLV657 | Immunoglobulin lambda |
| 28 | LOC652494 | — |
| 29 | DDX19A | ATP dependent RNA helicase involved in |
| | | mRNA export from the nucleus |

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### Examples

### Material and Methods

### Cell Lines and Tissue Samples

HeyA8 ovarian cell lines were grown at 37° C. with 5% CO₂ in MEM-EAGLE medium supplement with 2 mM L-glutamine, 1.5 g/L sodium bicarbonate, 4.5 g/L glucose, 10 mM HEPES, 1.0 mM sodium pyruvate and 10% fetal bovine serum (FBS).

qRT-PCR

Total RNA extracted from cell lines or lymphocytes was prepared with Trizol reagent according to manufacturer's protocol. RNA was subjected to Syber FAST ABI Prism qPCR Kit (KapaBiosystems Inc. Woburn, Mass., USA). Reactions were run on 7900HT Real Time PCR.

Relative expression levels of GFP variants were measured using the following primers:

| (SEQ ID NO: 2) | |
|---|---|
| FF: | 5′ TCTGTCTCCGGTGAAGGTGAAG 3′. |
| (SEQ ID NO: 3) | |
| Rev: | 5′ GGCATGGCAGACTTGAAAAAG 3′. |

Relative expression levels of BCL2 were measured using the following primers:

| (SEQ ID NO: 4) | |
|---|---|
| FF: | 5′ AGGCTGGGATGCCTTTGT 3′. |
| (SEQ ID NO: 5) | |
| Rev: | 5′ GACTTCACTTGTGGCCCAGATA 3′. |

Expression levels were normalized to the actin endogenous control.

### Construction of GFP-BCL2 Variants

GFP-BCL2 variants were obtained by introducing point mutations at the following locations in SEQ ID NO:1: +21A>G or +23G>A using appropriate primers set and the QuickChange II Site-Directed Mutagenesis Kit (Agilent Technologies) on the GFP-BCL2 variant template of Addgene (plasmid cat #3336). The mutations resulted in substitution of A to G at position 21 (rs1801018 variant) or G to A at position 23 (random variant) in the BCL2 mRNA product.

### Western Blot

HeyA8 Cells were seeded into 10 cm plates and transfected with GFP-empty vector, GFP-BCL2 wild-type, variant or a random. 48 hours following transfection, cells were washed with PBS and harvested in M2 lysis buffer (100 mM NaCl, 50 mM Tris, pH 7.5, 1% Triton X-100, 2 mM EDTA) containing protease inhibitor cocktail (Sigma). Extracts were clarified by centrifugation at 12,000×g for 15 min at 4° C., subjected to SDS-PAGE gel and proteins transferred to nitrocellulose membrane.

The membrane was blocked with 5% low fat milk and incubated with mouse anti-Bcl-2 (santa cruz) and rabbit anti-GFP (santa cruz) specific primary antibodies, washed with PBS containing 0.001% Tween-20 (PBST) and incubated with the appropriate horseradish peroxidase-conjugated secondary antibodies, mouse HRP and rabbit HRP, respectively. After washing in PBST, the membranes were subjected to enhanced chemiluminescence (ECL) detection analysis.

mRNA Stability

Heya8 cells transfected with GFP or one of three GFP-BCL2 variants (wild-type, +21 A>G or random +23 G>A), were treated 24 h post transfection with Actinomycin D (1 μg/ml, sigma) for 30 minutes at 37° C. Cells were examined at the following time points: 0, 2 h, 4 h and 7 h post incubation period. Total RNA was extracted using TriZol reagent.

### Cell Viability

Propidium iodide (PI) was added to the cell suspension at a concentration of 1 μg/ml. Sorting and analyses were carried out in a Gallios flow cytometer cell sorter collecting 10,000 events. Dead cells were excluded by gating on forward and side scatter and by eliminating PI-positive events.

### BCL2 Variant Response to Paclitaxel

Cells expressing GFP-empty vector or one of three types of GFP-BCL2 variants were measured for their sensitivity to paclitaxel. Following 24 hours of transfection with one of the indicated plasmids, cells were introduced to 100 or 200 nM of paclitaxel for 24 hours incubation.

### In Silico RNA Analysis

The RNA structure of the wild-type, variant and random BCL2 was predicted using the RNA folding prediction tool Mfold (Zuker, Nucleic Acids Research, 2003, Vol. 31, No. 13, 3406-3415). The significance of changes were quantified to present p-values using RNAsnp (Sabarinathan, Human mutation 34, 546, April 2013).

### Immunofluorescence

HEK293T cells were grown on coverslips in a 24-wells plate and transfected with wt-BCL2-GFP, variant-BCL2-GFP (+21 A to G in SEQ ID NO:1) or GFP empty vectors for 48 hours. Next, cells were fixed for 20 min in PBS containing 4% paraformaldehyde, washed 3 times with PBS, and permeabilized at the presence of 0.1% Triton X-100 for 10 min. Cells were incubated at room temperature with 4-6′ diamidino-2 phenylindole (DAPI) to stain cell nuclei. Cells were visualized using a confocal Microscope.

### Example 1: Paclitaxel Responsiveness in Cancer Patients is Highly Correlated with SNP rs1801018 Genotype

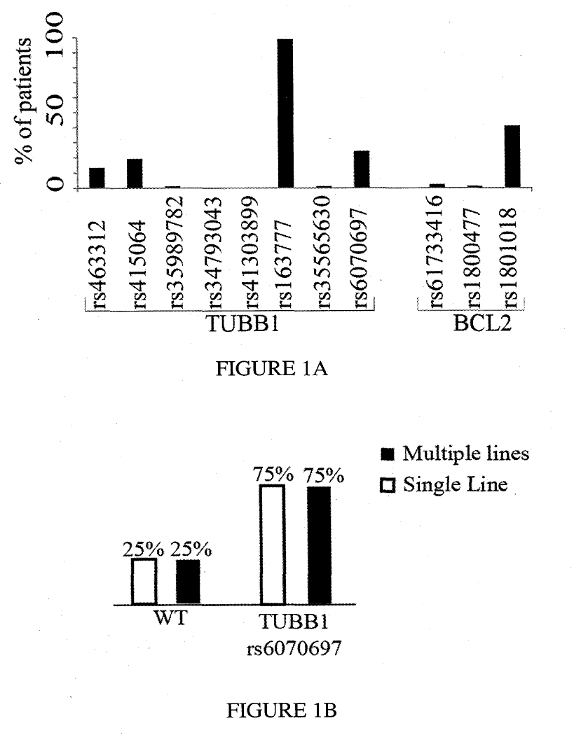

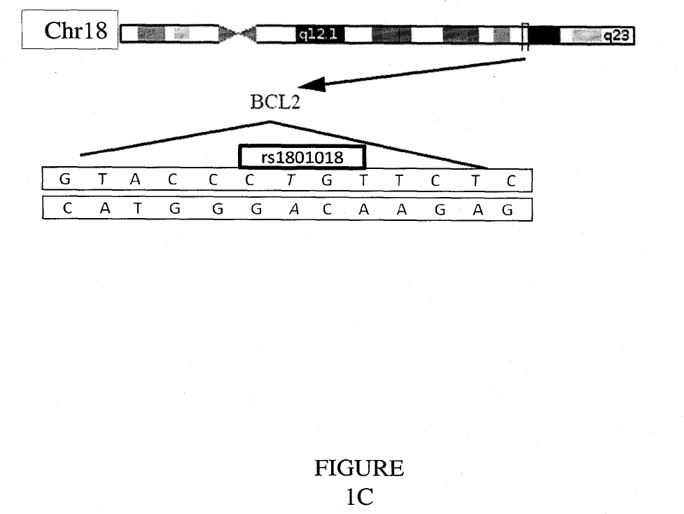

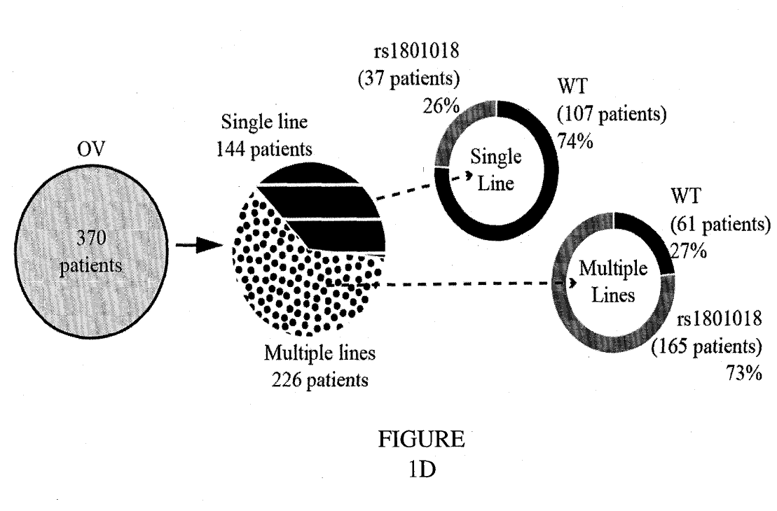

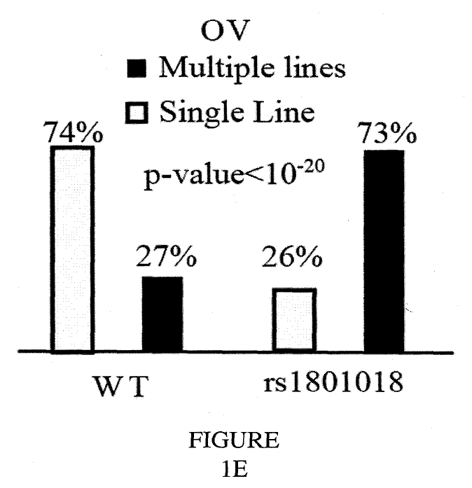

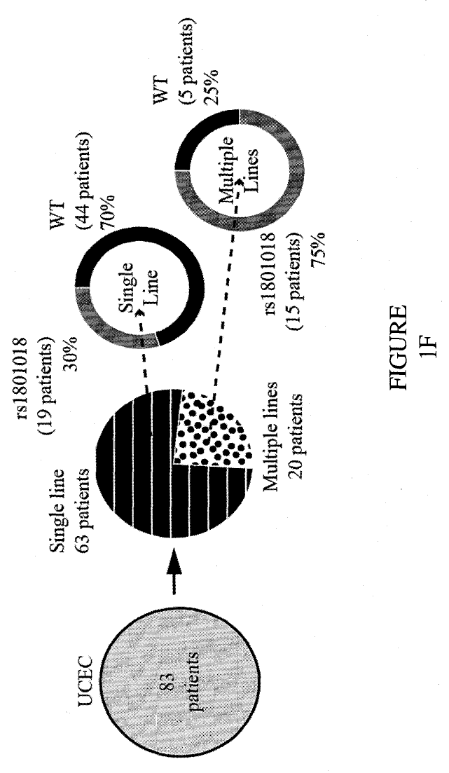

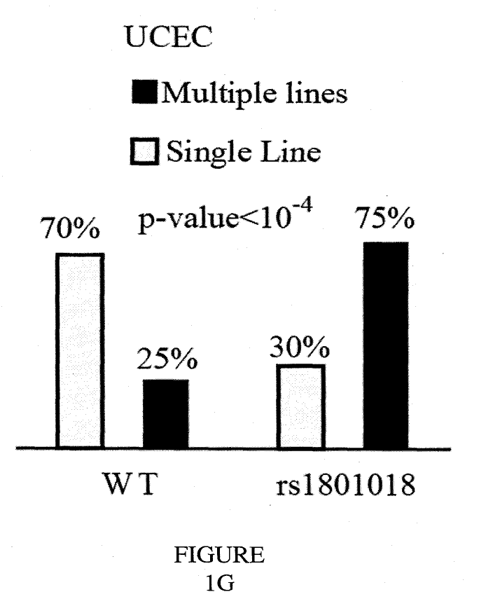

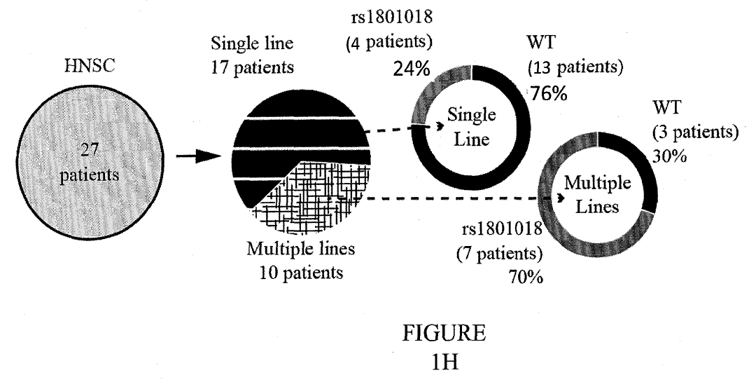

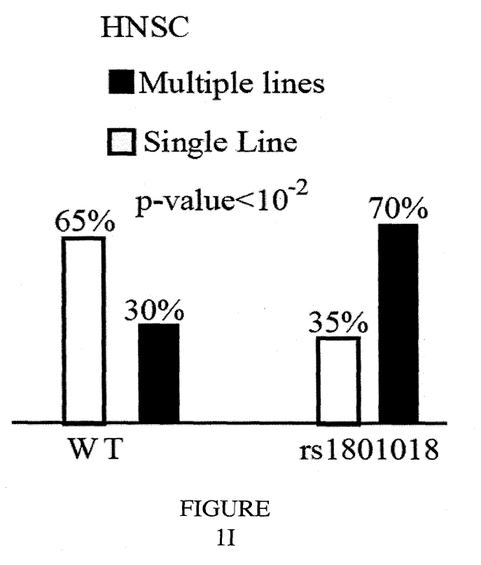

The correlation between responsiveness to paclitaxel treatment and single nucleotide polymorphism (SNP) was examined for BCL2 and TUBB1 known SNPs, since paclitaxel targets both genes. DNA samples of 566 cancer patients were screened for genotypic SNPs status. The cancer patients were grouped into patients who responded to paclitaxel (single line) or not (multiple line). As shown in FIG. 1A, a total of 11 SNPs were identified. Eight of the SNPs showed variation in less than 4% of patients, and one of the SNPs was present in 100% of the patients. Out of the two remaining SNPs, one (rs6070697) presented the same distribution in both groups (FIG. 1B), while the other (rs1801018) presented a significant association with the response to treatment. The SNP rs1801018, is a thymine to cytosine variant in location 63318646 on chromosome 18, which is location 5735 on the gene BCL2 (assembly GRCh38, build 106). Rs1801018 is a synonymous variant in the coding region of BCL2 (FIG. 1C).

The complete set of clinical data from The Cancer Genome Atlas (TCGA) included three types of cancer in which paclitaxel is used as a first line of treatment. These cancer types are Ovarian Cancer (OV), Uterine Corpus Endometrial carcinoma (UCEC) and Head and Neck Squamous Cell Carcinoma (HNSC). Response in these studies was defined according to whether or not patients have responded to first line of treatment. In OV, 144 patients have responded well to the first line of treatment, while 226 patients required additional lines of diverse chemotherapies. rs1801018 status is highly correlated with the affiliation to the first-line group versus the multiple-line group: out of the 226 patients who required additional lines of treatment, 73 percent (165 patients) displayed T in location 5735 of BCL2; 74 percent (107 patients) of the patients who required a single line of treatment displayed C in this location (FIGS. 1D and 1E). In the same manner, of 83 UCEC patients, 63 responded to the first line of treatment, while 20 patients require multiple lines. 70 percent of single-line responders displayed the wild type sequence, while 75 percent (15 out of 20) of multiple-lines patients displayed rs1801018 (FIGS. 1F and 1G). FIGS. 1H and 1I show that the stratification of the variant is consistent also in HNSC. The strong association is visible through the major differences in OV (FIG. 1E) between the high frequency of the variant in single-line (74%) versus multiple-line (27%) (p-value <10⁻²⁰), in UCEC (FIG. 1G) (p-value <10⁻⁴) and in HNSC (FIG. 1I) (p-value <10⁻²).

The results were further examined using additional types of cancer. Samples from a collection of 86 patients, from TCGA, with eight different cancer types which were treated with paclitaxel as a first line, were analyzed. As shown in FIG. 1J, the SNP was present in only 33% of patients affiliated with the single line group, 71% of patients in the multiple lines group were positive for the rs1801018 variant.

Overall, the results demonstrate a strong correlation between having a specific allele of SNP rs1801018 and the responsiveness to paclitaxel treatments.

### Example 2: The A to G rs1801018 Variant Leads to Significant Structural Changes in BCL2 mRNA Secondary Structure, which in Turn Results in a More Stable Transcript and Higher Bcl-2 Protein Level

Recent findings regarding the association of SNPs and cellular function indicate changes in RNA structural features as the main mechanism behind this association. Such structural features are fundamental in the function of RNA, and may be altered by the associated SNPs. A SNP may thus influence transcript stability and translation rate. RNA folding prediction software, using thermodynamic parameters, can be used for assessing the structural changes that occur around a SNP, compared to the wild-type structure.

The secondary structure was determined using predicted free energies. Two BCL2 SNPs (+21A>G vs. an adjacent random SNP +23G>A) were analyzed by the RNAsnp tool. The use of the tool has been performed using Mode 2 which predicts local changes in RNA secondary structure. The rs1801018 variant had a low p-value (p=0.0178) indicating a significant structural change of the variant (+21G) as compared to the wild-type BCL2 (+21A). The random SNP (+23 G>A) had high p-value (p=0.7337) which indicates non-significant structural changes as compared to the wild-type.

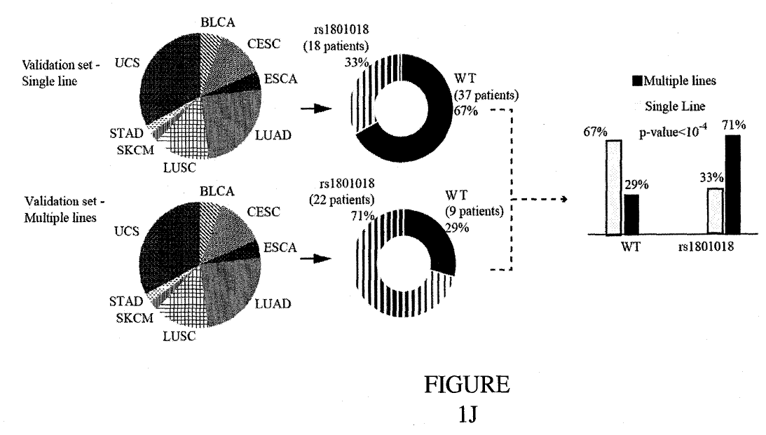

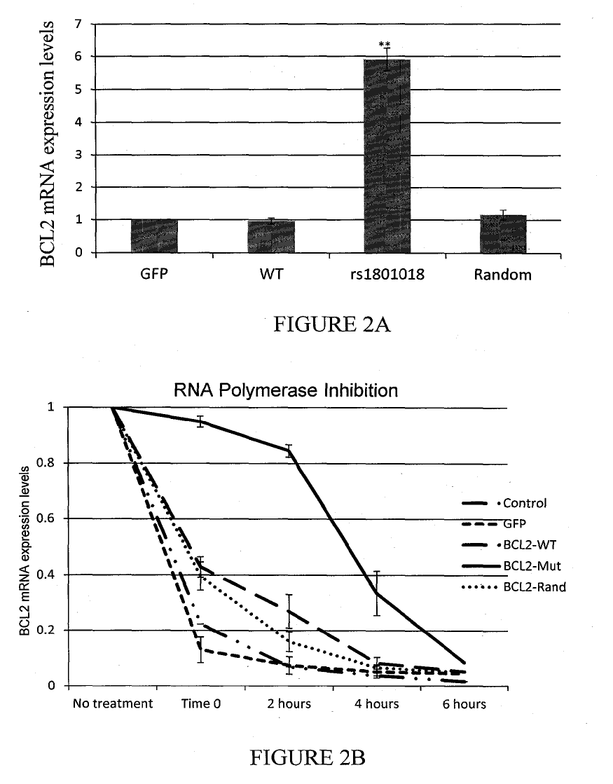

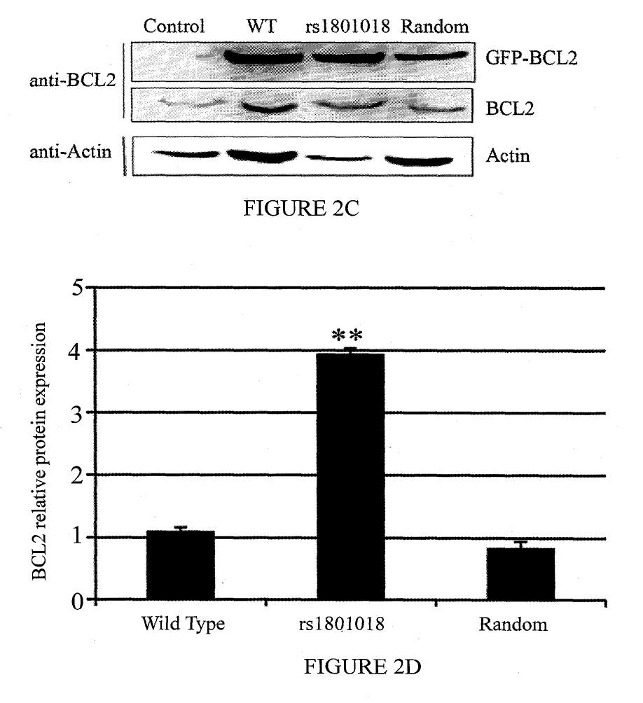

Next, the significant differences between the two variants were experimentally validated. HeyA8 cells were transfected with GFP-empty vector or one of 3 types of GFP-BCL2 variants (wild type, rs1801018 variant or random). Following incubation for 48 hours, total RNA was isolated from the cultured cells and served for cDNA synthesis. Relative transcript levels of GFP-BCL2 variants were measured by real-time qPCR (normalized to β-actin). As Shown in FIG. 2A, rs1801018 BCL2 amounts were significantly higher than other versions of the transcript.

The difference in stability of the transcripts was further evaluated using Actinomycin D treatment. HeyA8 cells were transfected with GFP-empty vector or one of 3 types of GFP-BCL2 variants as indicated. Following incubation for 24 h, transfected cells were incubated with 1 μg/ml of Actinomycin D for 30 minutes. Cells were harvested at the indicated time points and the total RNA was extracted. mRNA levels of GFP-BCL2 variants were determined by qPCR. FIG. 2B demonstrates that rs1801018 derived BCL2 (BCL2-Mut) produces a significantly more stable version of transcript, with an effect lasting 4 to 6 hours post actinomycin D treatment.

Next, the effect of SNP rs1801018 on Bcl-2 protein levels was examined. HeyA8 cells were transfected with GFP-empty vector or one of 3 types of GFP-BCL2 variants as indicated. 48 h later, cell lysates were subjected to SDS-PAGE gel and transferred to a nitrocellulose membrane. Anti-Bcl-2 detected both endogenous and GFP-Bcl-2. Actin measurement served as a loading control. GFP protein levels were quantified compared to Actin. As shown in FIGS. 2C and 2D, Bcl-2 protein expression was significantly higher in cells transfected with the SNP rs1801018 variant.

Overall, SNP rs1801018 is shown to affect the structure and stability of the BCL2 transcript, which further results in elevated amounts of the Bcl-2 protein.

### Example 3: A to G rs1801018 Variant Directly Affects Cells Sensitivity to Paclitaxel

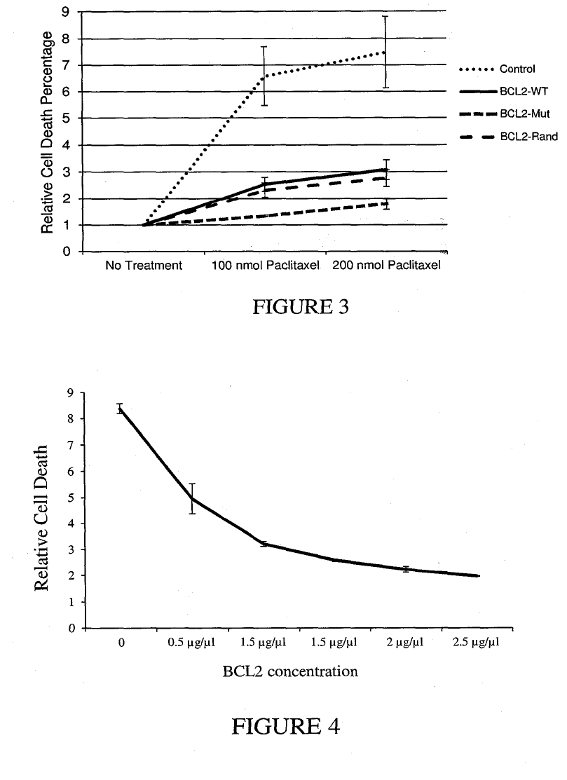

To examine the effect of rs1801018 variant on cells sensitivity to paclitaxel, GFP-empty vector control or one of three GFP-BCL2 variants (wild-type, rs1801018 variant or random) were over-expressed in cells, which then were tested with paclitaxel. Cells were incubated with increasing concentrations of paclitaxel as indicated in methods and in FIG. 3. FIG. 3 shows the response of three GFP-BCL2 variants (wild-type, rs1801018 variant (Mut) or random) or GFP-empty vector as control. Cells harboring GFP-empty vector (representing the intrinsic response of HeyA8 cells to paclitaxel) show high sensitivity to paclitaxel, as expected, due to the lack of over expressed BCL2. The response of cells transfected with either wild-type or random BCL2 led to reduced cell death (increased rates of cell viability), which is also expected, as levels of BCL2 are elevated. However, cell viability is significantly improved (p-value <0.05) upon transfection with the rs1801018 variant (BCL2-Mut). To study the relationship between resistance to paclitaxel and BCL2 gene copy numbers, a Bcl2 gradient assay was performed. The assay is designed to examine cell sensitivity to paclitaxel in the presence of increasing levels of BCL2 copies. HeyA8 cells were transfected with 5 different doses of wt-BCL2-GFP. 24 hours post transfection cells were treated with paclitaxel, and 24 hours post incubation cells viability was measured by FACS. As FIG. 4 shows, transfections with increased concentrations of BCL2 mRNA leads to decrease in relative cell death in response to paclitaxel.

To establish expression levels as the main cause for paclitaxel resistance and to rule out possible re-localization of the protein product of variant BCL2, spatial expression patterns of wild type versus variant BCL2-GFP was examined HEK293T cells were grown on coverslips in a 24-wells plate and transfected with wild-type BCL2-GFP, variant-BCL2-GFP (+21 A to G in SEQ ID NO:1) or GFP empty vector and applied for immunofluorescence assay. The staining of both wild type and variant BCL2-GFP demonstrated similar cell localization patterns whereas the empty vector was localized perfectly in cell nucleus.

1. A method of selecting an agent suitable for treating cancer in a subject, comprising: providing a sample of genetic material from the subject; and determining a single nucleotide polymorphism (SNP) rs1801018 allele in the sample of the subject, wherein homozygosity for the A allele at SNP rs1801018 is indicative of paclitaxel as a suitable anti-cancer agent, and the presence of at least one G allele at SNP rs1801018 is indicative of the need for an alternative agent as the suitable anti-cancer agent.

2. The method of claim 1, wherein the cancer is a solid cancer.

3. The method of claim 1, wherein the cancer is selected from the group consisting of ovarian cancer, uterine corpus endometrial carcinoma (UCEC), head and neck squamous cell carcinoma (HNSC), lung cancer, colon cancer, breast cancer, pancreatic cancer, prostate cancer, leukemia, and melanoma.

4.-5. (canceled)

6. The method of claim 1, wherein the cancer is a type of cancer considered amenable for treatment with paclitaxel.

7. The method of claim 1, wherein the selection of the alternative agent is based on further determination of an over-expression level of at least one gene listed in Table 1 in the subject compared to a non-cancer control subject.

8. The method of claim 7, wherein the selection of the alternative agent is based on further determination of a linear combination of the expression levels of at least two of the genes listed in Table 1 in the subject.

9. (canceled)

10. The method of claim 1, wherein the alternative anti-cancer is selected from the group consisting of antimetabolite, mitotic inhibitor, topoisomerase inhibitor, asparaginase, alkylating agent, antitumor antibiotic, topoisomerase II inhibitor, and combinations thereof.

11. The method of claim 1, wherein the alternative agent is docetaxel.

12. The method of claim 1, wherein the sample of the subject contains DNA or RNA.

13. The method of claim 1, wherein determining the allele at single nucleotide polymorphism (SNP) rs1801018 is by a technique selected from the group consisting of: analysis using a whole genome SNP chip, single-stranded conformational polymorphism (SSCP) assay, restriction fragment length polymorphism (RFLP), automated fluorescent sequencing; clamped denaturing gel electrophoresis (CDGE); denaturing gradient gel electrophoresis (DGGE), restriction enzyme analysis, chemical mismatch cleavage (CMC), RNase protection assays, use of polypeptides that recognize nucleotide mismatches, allele-specific PCR, sequence analysis, and SNP genotyping.

14.-16. (canceled)

17. A method of treating cancer in a subject in need of such treatment, comprising:
determining the genotype of a single nucleotide polymorphism (SNP) rs1801018 in a sample of the subject; and
(ii) administering paclitaxel to the subject when the sample is homozygous for the A allele of the SNP rs1801018, or administering alternative anti-cancer agent to the subject when at least one G allele of the SNP rs1801018 is present.

18. The method of claim 17, wherein the cancer is a solid cancer.

19. The method of claim 17, wherein the cancer is selected from the group consisting of ovarian cancer, uterine corpus endometrial carcinoma (UCEC), head and neck squamous cell carcinoma (HNSC), lung cancer, colon cancer, breast cancer, pancreatic cancer, prostate cancer, chronic myelogenous leukemia, and melanoma.

20.-21. (canceled)

22. The method of claim 17, wherein the cancer is a type of cancer considered amenable for treatment with paclitaxel.

23. The method of claim 17, wherein the alternative anti-cancer agent is docetaxel.

24. The method of claim 17, wherein the sample contains DNA or RNA.

25. The method of claim 17, wherein determining the allele at single nucleotide polymorphism (SNP) rs1801018 is performed by a technique selected from the group consisting of: analysis using a whole genome SNP chip, single-stranded conformational polymorphism (SSCP) assay, restriction fragment length polymorphism (RFLP), automated fluorescent sequencing; clamped denaturing gel electrophoresis (CDGE); denaturing gradient gel electrophoresis (DGGE), restriction enzyme analysis, chemical mismatch cleavage (CMC), RNase protection assays, use of polypeptides that recognize nucleotide mismatches, allele-specific PCR, sequence analysis, and SNP genotyping.

26.-31. (canceled)

32. The method of claim 17, wherein the method of treating further comprises a step of determining the expression of at least one gene listed in Table 1, wherein an alternative agent is administered when the gene is over-expressed compared to its expression in non-cancer subjects.

33. The method of claim 32, wherein the method of treating further comprises the step of determination a linear combination of expression levels of a plurality of genes listed in Table 1, wherein an alternative agent is administered when the plurality of genes is over-expressed in the subject compared to the expression in non-cancer subjects.

34.-45. (canceled)

46. The method of claim 1, wherein the method further comprises administering paclitaxel to the subject when the sample is homozygous for the A allele of the SNP rs1801018, or administering alternative anti-cancer agent to the subject when at least one G allele of the SNP rs1801018 is present.
